(19) United States
(12) Patent Application Publication
(10) Pub. No.: US 2007/0258896 A1
(43) Pub. Date: Nov. 8, 2007

(54) Contrast Agents In Medical Imaging

(75) Inventor: Ehud Nachaliel, Lower Galilee (IL)

Correspondence Address: WOLF, BLOCK, SCHORR & SOLIS-COHEN LLP, 250 PARK AVENUE, NEW YORK, NY, 10177, US

(73) Assignee: Mirabel Medical Systems Ltd., Migdal-HaEmek (IL)

(21) Appl. No.: 11/381,138

(22) Filed: May 2, 2006

(51) Int. Cl. A61K 49/00; A61B 5/00

(52) U.S. Cl. 424/9.1; 600/300

(57) ABSTRACT

A method of imaging a living body. The method includes administering a contrast agent to the body, sensing signals of a first modality, which does not use ionizing radiation, from the body, responsive to the contrast agent in the body and acquiring one or more images of at least a portion of the body using a second imaging modality different from the first modality, wherein at least one parameter of the acquiring of the one or more images is controlled responsive to the sensed signals of the first modality.

### FIELD OF THE INVENTION

The present invention relates to medical imaging and particularly to using contrast agents in medical imaging.

### BACKGROUND OF THE INVENTION

In computed tomography (CT), a CT-scanner illuminates a region of a person's body to provide images of internal structure and features in the region. The CT-scanner comprises an X-ray source that provides a fan or cone-shaped X-ray beam and an array of closely spaced X-ray detectors that face the X-ray source. The X-ray source and array of detectors are mounted in a gantry so that a person being imaged by the CT-scanner, generally lying on an appropriate support couch, can be positioned in a region, hereinafter a “field of view” (FOV), of the scanner, between the X-ray source and the detector array. The gantry and couch are moveable with respect to each other so that the FOV can be positioned axially at desired locations along an axial region of interest along the patient's body.

As exposing a patient to excess X-rays is undesirable, it is important to accurately time the CT imaging procedure to acquire images of an imaged body region, with minimal x-ray exposure.

In many instances, differences in X-ray absorption of different tissues in the body are not sufficient to provide CT-images of a sufficient diagnostic quality. In some cases, a contrast agent is introduced by a physician into the patient's body to improve contrast in the imaged body region. The contrast agent is generally administered at a point remote from the imaged body region and it is important to accurately time the CT scan with the time at which the contrast agent maximally enhances contrast in the imaged body region. If the timing is not accurate, there may be a need to repeat the contrast agent administration and the CT imaging. It is noted that for some contrast agents, a specific amount of the contrast agent should be achieved, and larger or smaller amounts do not provide images of sufficient quality.

U.S. patent publication 2005/0228273 to Tamakoshi, the disclosure of which is incorporated herein by reference, describes a system for CT scanning and injecting a contrast agent.

Generally, a time window in which the imaging should be performed is relatively short and it is usually difficult to accurately determine when the contrast window begins or ends. In some cases, the duration and timing of the contrast window is a function, inter alia of the specific body organ being imaged, the patient's age, sex, size, cardiac output and hydration, the specific contrast agent and the administration protocol of the contrast agent. It can therefore be difficult to accurately synchronize the operation of the CT-scanner with the introduction of the contrast agent.

A number of different strategies have been developed to synchronize a contrast window with a CT-scan. In some protocols, a relatively small “test” dose of contrast agent is introduced into a patient's body and a number of CT-scans, referred to as “pre-scans”, of the patient are performed by the CT-scanner using a relatively low X-ray intensity. The low intensity pre-scans are used to track uptake of the contrast agent by the body organ of interest and indicate how long after administration of the contrast agent its concentration in the body organ is expected to be optimal. A full dose of the contrast agent is then administered to the patient and an imaging CT-scan of the patient at full X-ray intensity is performed with a timing controlled responsive to results provided by the pre-scans.

The use of pre-scans includes using additional X-rays and also requires high skill from the operator.

Similar problems of timing image acquisition with injection of a contrast agent appear with other imaging modalities, such as magnetic resonance imaging (MRI).

U.S. patent publication 2003/0158476 to Takabayashi, the disclosure of which is incorporated herein by reference, describes an MRI system which provides monitoring scans before performing an imaging scan, in order to allow a physician to time the scanning with body intake of a contrast agent.

U.S. patent publication 2003/0158476 to Prince, the disclosure of which is incorporated herein by reference, describes a method of timing MRI image acquisition with contrast agent delivery.

These and other methods may improve the timing but they are complex and still do not provide sufficient improvement.

### SUMMARY OF THE INVENTION

An aspect of some embodiments of the invention relates to controlling a medical imaging process (e.g., a medical scan) of a first modality responsive to measurements of a second modality, which does not use ionizing radiation. Optionally, the second modality comprises electrical measurements, generally referred to herein as impedance measurements. Electrical impedance measurements are relatively inexpensive, do not use ionizing radiation and are fast, and therefore provide a fast and safe method for acquiring information which can be used for better control of the medical imaging process.

In some embodiments of the invention, the measurements of the second modality track uptake of a contrast agent within the patient and the imaging process is controlled accordingly. Alternatively or additionally, the measurements of the second modality are used to select a region of interest to be imaged by the imaging process.

The measurements of the second modality may include forming an image, for example a surface image or a cross-section image, or determining impedance values at one or more points without generating an image.

In some embodiments of the invention, the information from the measurements of the second modality is used to time the medical imaging using the first modality. Optionally, a start time of a medical imaging scanning procedure is adjusted responsive to the measurements of the second modality. Alternatively or additionally, a rate of the scanning procedure, such as a speed or movement profile of translation of a patient couch in CT imaging and/or an angular velocity of an X-ray source of a CT imager is controlled responsive to the measurements of the second modality. Further alternatively or additionally, the information from the measurements of the second modality is used to control a voltage, current or other parameter of radiation signals (e.g., X-rays) directed at the body portion being imaged. Further alternatively or additionally, one or more parameters of the image acquisition apparatus are adjusted responsive to the measurements of the second modality.

Optionally, the control of the imaging using the first modality responsive to the measurements of the second modality is performed automatically by a controller, without human intervention. Alternatively or additionally, the measurements of the second modality are used to provide information which is presented (e.g., displayed, sounded) to a human operator. The operator then controls the imaging based on the presented information.

In some embodiments of the invention, sensing elements of the second modality (e.g., electrodes used in electrical impedance measurement) are formed from a material which is relatively transparent and/or uniform to the first modality, such that the sensing elements can be placed close to the imaged body area, without substantially interfering with the imaging. In an exemplary embodiment of the invention, the sensing elements are formed of a low-density conductive polymer that is relatively transparent to X-rays, for use with CT x-ray imaging. Alternatively or additionally, the sensing elements are placed on the patient outside the imaged body region. A controller optionally adjusts the results of the measurements of the second modality responsive to the distance between the sensing elements and the imaged region.

There is therefore provided in accordance with an exemplary embodiment of the invention, a method of imaging a living body, comprising administering a contrast agent to the body, sensing signals of a first modality, which does not use ionizing radiation, from the body, responsive to the contrast agent in the body and acquiring one or more images of at least a portion of the body using a second imaging modality different from the first modality, at least one parameter of the acquiring of the one or more images is controlled responsive to the sensed signals of the first modality. Possibly, the second imaging modality comprises CT imaging or MRI. Optionally, the second imaging modality comprises nuclear medicine imaging. Optionally, sensing signals of the first modality comprises sensing electrical signals. Optionally, sensing the electrical signals comprises sensing through an array of sensing electrodes including at least two rows and two columns of electrodes. Optionally, sensing the electrical signals comprises sensing using a linear array of sensing electrodes. Optionally, sensing the electrical signals comprises sensing using at least 10 sensing electrodes. Optionally, sensing signals of the first modality comprises sensing using a probe which is substantially transparent to a radiation used by the second imaging modality.

Optionally, the method includes generating a map of an area of the body responsive to the sensed signals of the first modality and the at least one parameter of the acquiring of the one or more images is controlled responsive to the generated map.

Optionally, the method includes displaying the map. Optionally, the map comprises an impedance map. Optionally, the method includes determining a spatial concentration of the contrast agent in a plurality of points on the body and the at least one parameter of the acquiring of the one or more images is controlled responsive to the determined spatial concentration. Optionally, the at least one parameter comprises a time parameter of the acquiring of the images using the second modality. Optionally, the acquiring of the one or more images is initiated responsive to the sensed signals of the first modality.

Optionally, the acquiring of the one or more images is terminated responsive to the sensed signals of the first modality. Optionally, a rate of the acquiring of the one or more images is controlled responsive to the sensed signals of the first modality.

Optionally, acquiring of the one or more images comprises radiating the patient and an intensity of the radiation is selected responsive to the sensed signals of the first modality. Optionally, the method includes analyzing the sensed signals of the first modality to determine a medical diagnosis of the body.

There is further provided in accordance with an exemplary embodiment of the invention, a method of imaging a living body, comprising sensing electrical signals from the body, acquiring one or more images of at least a portion of the body using an imaging modality other than impedance imaging, at least one parameter of the acquiring of the one or more images is controlled responsive to the sensed electrical signals.

Optionally, the at least one parameter comprises a boundary of a region from which the images are acquired. Optionally, the at least one parameter of the acquiring of the one or more images is controlled automatically by a processor responsive to the sensed electrical signals.

Optionally, the at least one parameter of the acquiring of the one or more images is controlled by a human responsive to a display of a representation of the sensed electrical signals. Optionally, the at least one parameter of the acquiring of the one or more images is controlled responsive to a low quality image generated based on the sensed electrical signals. Optionally, the at least one parameter of the acquiring of the one or more images is controlled responsive to an attribute of a contrast agent in the body determined based on the sensed electrical signals.

There is further provided in accordance with an exemplary embodiment of the invention, a medical imaging apparatus, comprising one or more electrodes adapted for acquiring electrical signals from a subject, an imaging unit adapted to acquire medical images of the subject, using a modality other than electrical impedance imaging and a controller adapted to generate an indication of a desired timing of the imaging unit, responsive to electrical signals acquired by the one or more electrodes.

Optionally, the one or more electrodes are substantially transparent to the modality of the imaging unit. Optionally, the one or more electrodes comprises at least six electrodes. Optionally, the apparatus includes a syringe adapted to administer a contrast agent to the subject under control of the controller. Optionally, the controller is adapted to determine information on a contrast agent in the subject and accordingly generate the indication of the desired timing. Optionally, the controller is adapted to generate an indication of a desired time at which to begin an imaging session. Optionally, the controller is adapted to generate an impedance image, for example an electrical impedance tomographic image.

There is further provided in accordance with an exemplary embodiment of the invention, a medical imaging apparatus, comprising one or more electrodes adapted for acquiring electrical signals from a subject, an imaging unit adapted to acquire medical images of the subject using a modality other than electrical impedance imaging and a controller adapted to determine information on a contrast agent in the subject and accordingly provide suggested values for one or more parameters of the operation of the imaging unit. Possibly, the one or more parameters comprise a beginning time of an imaging session of the imaging unit.

### BRIEF DESCRIPTION OF FIGURES

Non-limiting examples of embodiments of the present invention are described below with reference to figures attached hereto. In the figures, identical structures, elements or parts that appear in more than one figure are generally labeled with a same numeral in all the figures in which they appear. Dimensions of components and features shown in the figures are chosen for convenience and clarity of presentation and are not necessarily shown to scale. The figures are listed below.

FIG. 1 schematically shows a CT-scanner system comprising an electrical impedance scanner, in accordance with an embodiment of the present invention;

FIG. 2 is a flowchart of acts performed during an imaging session, in accordance with an exemplary embodiment of the invention;

FIG. 3 is a schematic block diagram of an imaging system, in accordance with an exemplary embodiment of the invention; and

FIG. 4 is a schematic graph of the concentration of a contrast agent as determined by an EIS-scanner, in accordance with an exemplary embodiment of the invention.

### DETAILED DESCRIPTION OF EXEMPLARY EMBODIMENTS

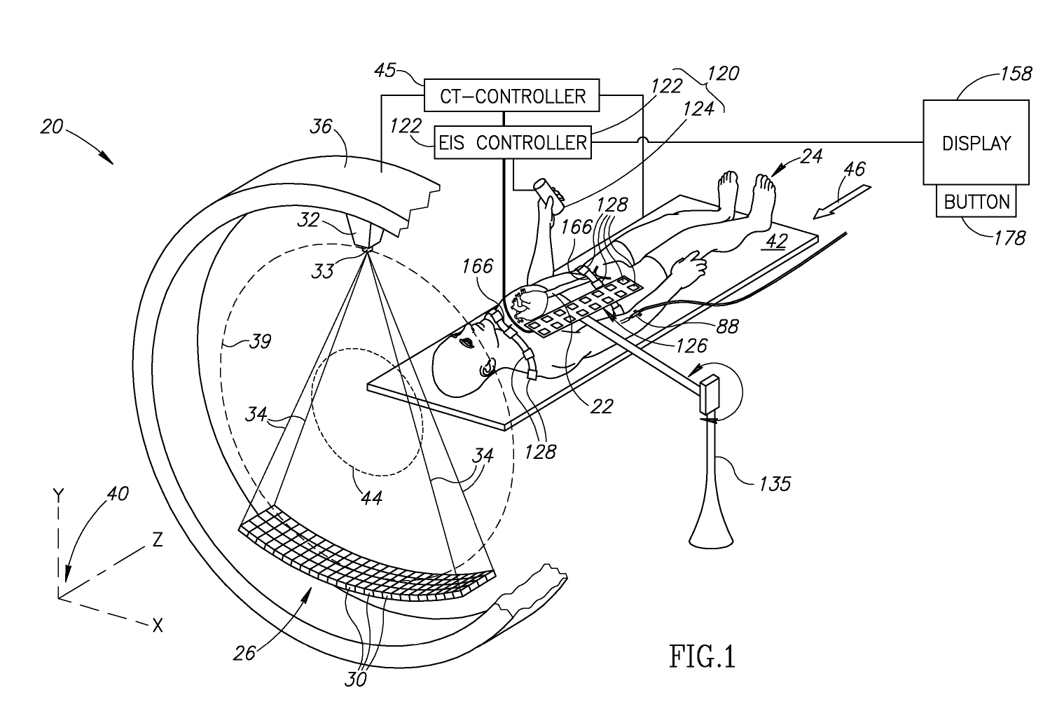

FIG. 1 schematically shows a CT scanner system 20 comprising an electrical impedance scanner (EIS-scanner) 120, in accordance with an exemplary embodiment of the present invention. By way of example CT scanner system 20 is shown performing a CT-scan of the abdominal aorta 22 of a patient 24. In performance of the CT-scan, a bolus of a contrast agent is administered to the patient to contrast abdominal aorta 22 against the background of surrounding tissue. A syringe 88 is assumed to be used to administer the bolus of contrast agent to the patient. EIS-scanner 120 is used to image and track the contrast agent and determine when its concentration in a region of aorta 22 is optimum for imaging the aorta region. Aspects of performance of the CT-scan are controlled, in accordance with an embodiment of the invention, responsive to information provided by EIS-scanner 120. Only features of scanner system 20 and EIS-scanner 120 germane to the discussion are shown in FIG. 1.

CT Scanner

Optionally, CT-scanner system 20 comprises a multislice CT scanner including a detector array 26 having X-ray detectors 30 and an X-ray source 32. X-ray source 32 provides a cone beam of X-rays schematically outlined by lines 34 for illuminating patient 24 with X-rays that are radiated from a focal spot 33 of the X-ray source. X-ray source 32 and detector array 26 are mounted to a rotor 36 (shown partially cutaway) of a gantry (not shown) comprised in CT-scanner system 20. Rotor 36 is rotatable around the z-axis of a coordinate system 40 in a “rotation plane” schematically indicated by a dashed circle 39 generally perpendicular to the z-axis so as to position X-ray source 32 and detector array 26 at different view angles about the z-axis. Patient 24 is supported on a couch 42 during imaging of the patient. Couch 42 is controllable to be translated axially along the z-axis (e.g., in a direction indicated by arrow 46) to move patient 24 through a field of view (FOV), schematically indicated by a dashed circle 44, of scanner system 20. A CT-controller 45 controls the operation of the CT scanner, for example, the motion of couch 42, the motion of rotor 36 and/or the current and/or voltage of X-rays provided by X-ray source 32.

EIS Scanner

EIS-scanner 120 comprises an EIS-controller 122, at least one source electrode 124, optionally held in the hand of patient 24, and sensor electrodes 128 attached to the patient's skin using any of various techniques and devices known in the art. EIS-controller 122 controls application of current and/or potential to source electrode 124 and reception of current and/or potential signals from sensor electrodes 128 responsive to the current and/or potential applied to source electrode 124. EIS-controller 122 optionally processes the signals that it receives from sensor electrodes 128 to determine concentration of the contrast agent in the body of patient 24, in the vicinity of the imaged organ. A display 158 optionally provides indications to a physician. Various designs of electrode arrays are shown in FIG. 1. These can be useful for determining impedance in various regions of the body.

In some embodiments of the invention, EIS scanner 120 is designed for electrical impedance imaging, for example surface electrical imaging such as described in U.S. Pat. No. 5,810,742 to Pearlman and/or in U.S. Pat. No. 6,560,480 to Nachaliel et al., the disclosures of which patents are incorporated herein by reference. For example, an array 126 may include a large number, e.g., at least 40, at least 60 or even at least 100, of sensor electrodes 128, which allow achievement of sufficient image resolution. In some embodiments of the invention, an array of electrodes, for example an array including at least 2 rows and at least 3 or even 5 columns, is used to sense electrical signals for the impedance measurements. In an exemplary embodiment of the invention, an array of at least 8×8 electrodes each sensing separate electrical signals is used for the impedance measurement. EIS scanner 120 possibly includes one or more peripheral electrodes which serve as guard rings.

In FIG. 1, sensor electrodes 128 of array 126 are positioned in a relatively long narrow array 126 that extends along a length of the body of patient 24 to correspond to the position and orientation of abdominal aorta 22. However, other array arrangements may be advantageous. Optionally, a spatial configuration of electrodes 128 is tailored to an imaging task of the CT-scan. For example, for CT-imaging of a specific organ such as the liver, EIS electrodes are positioned in a relatively localized region of the body surface near the liver. For CT-angiography of the cardiovascular system, a linear array of electrodes that extends along a substantial length of the patient's body may be used.

Alternatively or additionally to surface electrical images, the impedance images generated by EIS scanner 120 are tomography images which show the impedance of a slice of the patient's body. These embodiments are optionally used with CT images directed at cross-sectional details, such as the aorta. One or more belts 166 including sensors 128 are optionally mounted around the patient's body. The signals sensed by the sensors 128 of the belts 166 are used to generate tomography impedance images. In some embodiments of the invention, sensors 128 are placed symmetrically around belt 166, so as to minimize artifacts caused by the electrodes in the CT images, if belt 166 is placed within the area covered by the CT scan. In the embodiment of FIG. 1, both array 126 and belts 166 are used, but this is not necessary.

Alternatively to generating an image, EIS scanner 120 is not capable of generating an image or is operated in a mode which does not generate an image. Possibly, EIS scanner 120 determines an impedance level at a single point or calculates an average impedance level at a plurality of points on patient 24. In some embodiments of the invention, in accordance with this alternative, array 126 includes fewer than 20, fewer than 10 or even not more than 6 sensing electrodes 128. In an exemplary embodiment of the invention, EIS scanner 120 includes only a single sensor electrode 128. In other embodiments of the invention, electrical measurements are acquired at a plurality of sensing points and the measurements are compared without generating an image. The measurements at the plurality of sensing points are optionally used to estimate a progression time of the contrast agent.

It is noted that while only a single source electrode 124 is shown, a plurality of source electrodes may be used. In an exemplary embodiment of the invention, an array similar to array 126 of sensing electrodes 128, possibly located parallel to array 126, is used to apply the electrical signals. Optionally, a spatial configuration of the electrodes for applying electrical signals is tailored to an imaging task of the CT-scan. In some embodiments of the invention, one or more of electrodes 128 of array 126 is used instead of source electrode 124 to provide electrical signals to the patient.

Transparency of Electrodes

Sensor electrodes 128 are formed, in some embodiments of the invention, so that they are substantially transparent to X-rays provided by X-ray source 32, i.e., the sensor electrodes does not substantially interfere with the CT imaging. Optionally, to increase transparency of sensor electrodes 128 the electrodes are made relatively thin and/or are formed of a material, such as a polymer, that has a relatively small interaction with the X-rays. In some embodiments of the invention, transparent electrodes in accordance with any of the embodiments described in U.S. patent application Ser. No. 10/296,010, published as US patent publication 2004/0077944, the disclosure of which is incorporated herein by reference, are used.

In some embodiments of the invention, at least one of the electrodes is formed so that it is relatively clearly imaged by X-rays from X-ray source 32. The at least one electrode functions as a fiducial electrode and its location on the body relative to features of the body imaged by the CT-scanner can be relatively well defined from CT-images provided by the scanner. Optionally, the location of the at least one “fiducial” sensor electrode is used to determine the location of other sensor electrodes 128 relative to features of the body imaged by the CT-scanner. In some embodiments of the invention, a fiducial mark is included on a belt or other array housing connecting electrodes 128.

Operation

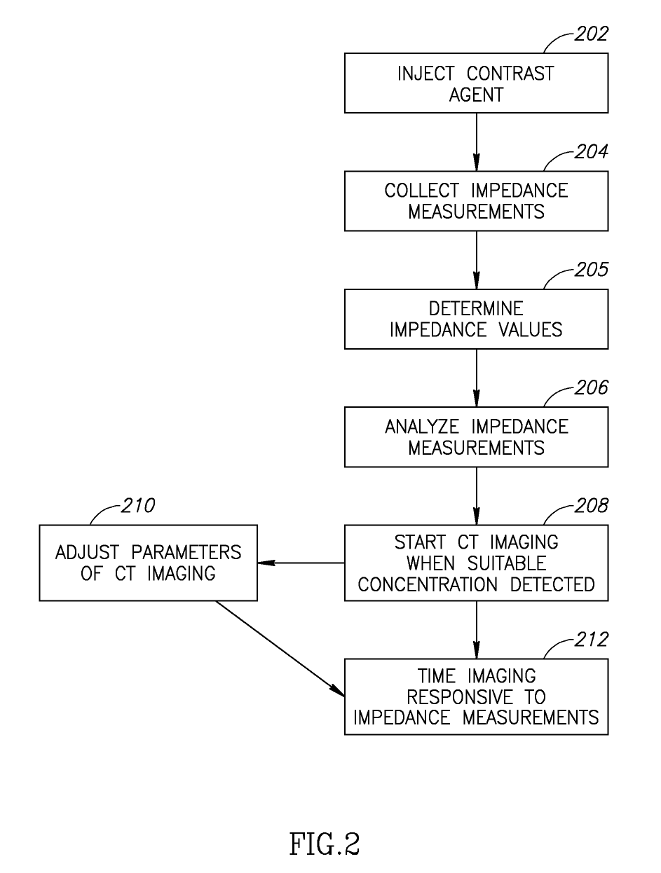

FIG. 2 is a flowchart of acts performed during an imaging session, in accordance with an exemplary embodiment of the invention. When the imaging procedure is to begin, a contrast agent is injected (202) into the patient. Electrical signals are then collected (204) from the patient using EIS-scanner 120 and used to determine (205) impedance values. The impedance values are analyzed (206) to determine when the contrast agent has a suitable concentration for CT imaging and the CT imaging is started (208) at a time selected according to the analysis. In some embodiments of the invention, one or more parameters of the CT imaging are adjusted (210) responsive to the impedance imaging results. Optionally, EIS scanner 120 continues determining the impedance throughout the CT imaging. In some embodiments of the invention in accordance with this option, the CT imaging is timed (212) and/or terminated according to impedance results acquired during the CT imaging.

In other embodiments of the invention, once the CT imaging begins, the operation of EIS scanner 120 is stopped so that it does not interfere with the CT imaging. Optionally, array 126 is removed from the patient before the CT imaging begins, for example when couch 42 begins to move. In some embodiments of the invention, CT scanner system 20 includes an arm 135 which automatically removes EIS scanner 120 from patient 24, for example upon instructions from EIS controller 122.

Contrast Agent

Referring in more detail to injecting (202) the contrast agent, in some embodiments of the invention the contrast agent comprises iodine or an iodized salt, such as Iopromide—ultravist 300. Alternatively or additionally, the contrast agent comprises gadolinium or any other contrast agent which is suitable for X-ray and is detectable in impedance values. Further alternatively or additionally, any of the contrast agents (or mixtures thereof) described in one or more of U.S. Pat. No. 5,651,955 to Klaveness, U.S. Pat. No. 5,733,525 to Klaveness, U.S. Pat. No. 6,416,740 to Unger, U.S. Pat. No. 6,558,665 to Cohen et al. and U.S. Pat. No. 6,797,257 to McDonald et al., the disclosures of which are incorporated herein by reference, which is detectable in impedance measurements and enhances the contrast and/or quality of the controlled CT scan or other imaging modality, is used.

Optionally, the entire dose of the contrast agent is injected at once. Alternatively, the contrast agent is injected in a plurality of separate reduced doses. In some embodiments of the invention, after injection of a first dose, the results of the impedance values are analyzed to determine how much more is to be injected. Alternatively or additionally, if during the CT imaging the level of the contrast agent falls below a predetermined level, an additional dose of the contrast agent is added to the patient. A notice on a suggested additional amount of the contrast agent and/or on the need to add more contrast agent is displayed to the physician, for example on display 158. Alternatively, the injection of the contrast agent is controlled automatically by EIS controller 122.

Location of Electrical Sensors

Referring in more detail to collecting (204) the electrical signals, in some embodiments of the invention, the electrical signals are acquired from the location being imaged in the CT imaging and/or very close thereto, and the CT imaging begins immediately when instructions to begin are given by EIS controller 122. In these embodiments, the analysis is directed at determining when the impedance values determined from the sensed signals are indicative of a concentration of the contrast agent which is suitable for CT imaging.

In other embodiments of the invention, the electrical signals are not acquired at the CT imaged location, but rather at a location adjacent, or remote from, the location imaged by the CT scanner. Optionally, the analysis on when to perform the CT scanning is based on a spatial interpolation or extrapolation from the location at which the sensed signals are acquired, to the CT scanning area. In some embodiments of the invention, electrical signals are acquired on opposite sides of the CT scanned area. For example, two impedance measurement electrodes 128 or two belts 166 may be placed at opposite sides of the axial range of interest in which the CT scanning is performed (as illustrated by belts 166 in FIG. 1), outside the imaged area. For example, the belts 166 of electrodes may be placed on opposite sides, along the height of the patient (in the Z direction), of the imaged area. The impedance values from the opposite sides of the CT imaged area are used in estimating the concentration of the contrast agent in the imaged area.

In some cases, electrodes 128 used for sensing electrical signals are located only on one side of the axial area of interest. Optionally, in these cases, the impedance in the area of interest is determined based on extrapolation from a mapping of the impedance values acquired by EIS controller 122. Alternatively or additionally, any other prediction method is used to decide when to perform the CT imaging.

Values Used

The determined impedance values are optionally the real conductivity or resistance of the tissue, for example, based on the relation between the applied voltage and measured current. Alternatively or additionally, any other impedance measures, such as the complex impedance, or any of the impedance measures related to in, above mentioned, U.S. Pat. No. 5,810,742 to Pearlman, is used in the analysis. In an exemplary embodiment of the invention, a frequency at which the impedance is lowest or highest or a frequency map of the impedance is used.

The electrical signals are optionally collected at a single frequency, which provides best contrast for the task. The frequency may be determined in clinical tests on a plurality of patients. In some embodiments of the invention, the frequency used is of between 5-100 KHz, in order to overcome the effects of skin, although other frequencies may be used. Possibly, the measurements are performed at a plurality of frequencies and the best results are used. Alternatively or additionally, the signals are sensed at two frequencies and the difference in the impedance between the two frequencies is used in tracking the contrast agent. For example, a frequency with an impedance value highly related to effects unrelated to the contrast agent, such as skin impedance, may be used together with a frequency which is only slightly affected by skin impedance.

Further alternatively or additionally, the directly measured current or voltage is used, for simplicity, without calculating an impedance. In accordance with this alternative, the measured current or voltage is the impedance value.

Analysis

The determined impedance values are optionally used to generate an impedance map (e.g., surface map, tomography map) of the patient. In the analysis (206), a blood vessel of interest for the CT scanning is identified and the average impedance of the blood vessel is determined. Possibly, the impedance of the blood vessel is normalized to an average of the impedance of the map to overcome measurement related variations, for example variations in the contact of the electrodes 128 with the patient's skin.

In other embodiments of the invention, in which electrical signals are collected through a plurality of electrodes, the impedance value used in determining when the contrast agent has a suitable concentration is an average of the impedance values of the pixels of the generated map, possibly excluding up to a predetermined number and/or percentage (e.g., 10%, 20%) of outlier values. Alternatively or additionally, a median impedance value and/or a highest or lowest impedance value is used in the analysis.

While in the above exemplary embodiments a single impedance value is determined from the entire impedance map at any specific time, it is possible that a plurality of values be used from a single map, for example when a blood vessel covers a substantial portion of the map and/or when the changes in impedance are clearly identifiable on the map. Optionally, in these cases, the impedance values of a plurality of points on the map are used to follow the contrast agent, for example to extrapolate to a point of interest not on the map.

Timing Issues

Referring in more detail to analyzing (206) the impedance values, in some embodiments of the invention, the CT imaging is started (208) when the impedance is lower than a predetermined threshold or otherwise meets a predetermined condition. Alternatively or additionally, the CT imaging is started when the changes in the impedance value meet a predetermined requirement, for example when a decrease rate of the impedance goes below a predetermined value. Possibly, the CT scan begins when a maximal concentration of the contrast agent is reached or at a time that provides that the maximal concentration is expected to be reached in the middle of the CT scan. Thus, in accordance with some embodiments, the analysis (206) relates to variations of the impedance values over time. The specific criteria for starting (208) the CT scan is optionally selected according to the specific contrast agent used, the specific patient details and/or the accuracy of the impedance measurements.

In some embodiments of the invention, clinical tests are performed on a plurality of patients in order to determine the optimal time for initiating the CT imaging. Optionally, in the clinical tests, CT images are acquired over a span of time in parallel to recording the impedance values collected by impedance scanner 120. Accordingly, the conditions on the impedance values under which the CT scanning is initiated are determined. These values are configured into controller 122 at manufacture. Alternatively or additionally, each CT scanner 120 is calibrated separately at installment and/or periodically, based on correlation between CT scans and impedance values of a predetermined number of patients. Optionally, controller 122 continually updates the conditions for initiation of the CT scanning according to feedback from the physician and/or automatic analysis of the CT images. For example, the acquired CT images may be checked to determine cases in which the earliest or latest CT images are of insufficient quality, e.g., have low contrast between different types of tissue. If the same problem repeats, the condition on the impedance measurements that determines the timing of the imaging is optionally changed to avoid the problem. In the case of physician feedback, the initiation conditions are optionally adjusted when the physician consistently indicates too early or too late initiation.

Preliminary Procedure

In some embodiments of the invention, a preliminary procedure with a limited dose of contrast agent injected into the patient, is carried out using EIS scanner 120, before the CT imaging begins. In the preliminary procedure, a complete profile of the changes in impedance due to the contrast agent are determined in the specific patient for which the CT imaging is to be performed. Accordingly, the dose of contrast agent required, the time at which the CT scanning is to begin and/or other parameters of the procedure, such as the voltage, current or other parameters of the CT scanner are determined. Thereafter, a complete dose of the contrast agent is injected into the patient and the CT imaging is performed using the determined parameters.

In an exemplary embodiment of the invention, the impedance values responsive to the limited dose of the contrast agent are used to determine when a maximum or minimum of the impedance value is expected and accordingly the time at which the scan is begun is selected.

Possibly, in the preliminary procedure, a less toxic contrast agent is used, relative to the contrast agent used during the CT scan. In some embodiments of the invention, a more diluted contrast agent is used in the preliminary procedure. Alternatively, the same contrast agent is used for the CT scan and for the preliminary procedure.

In some embodiments of the invention, one or more parameters of EIS scanner 120 are adjusted responsive to the results of the preliminary procedure.

Starting CT Scan

In some embodiments of the invention, controller 122 displays (e.g., on display 158) an indication to the physician when the CT scan is to begin. For example, an orange light is lit when the beginning of the CT scan is considered premature and a green light is lit when the beginning of the CT scan is recommended. In some embodiments of the invention, instead of using a separate light for indicating the contrast level is as desired and for indicating that the CT system is ready, the ready to start light of CT systems known in the art is only lit when the contrast level is considered optimal for the CT scan. Alternatively or additionally, instead of a simple yes/no (e.g., red/green) indication, an impedance value is displayed, a graph of the change of the impedance value is displayed and/or the impedance map is displayed to the physician, who determines when to begin the CT scan accordingly. A button 178 or any other control is optionally used by the physician to begin the CT scan. Alternatively, controller 122 automatically begins the CT scan, without user intervention, when the impedance measurements reach the predetermined conditions.

Optionally, the CT imaging begins immediately when an instruction is given by controller 122. Alternatively, from the time at which it is determined that the CT imaging is to be performed a substantial amount of time (e.g., more than 10-20 seconds) passes until the CT imaging actually begins. In accordance with this alternative, the condition(s) for initiating the CT imaging are set to initiate the imaging a required time before the optimal distribution of the contrast agent is expected to be achieved.

Other Parameters

As mentioned above, in addition, or alternatively, to timing the beginning of the CT imaging, in some embodiments of the invention, controller 122 times (212) the duration of the CT imaging and/or the end time of the CT imaging. For example, the CT scan may be stopped when the concentration of the contrast agent, as determined from the impedance values, goes below a predetermined value, which makes the imaging ineffective. This may reduce substantially the amount of radiation to which the patient is subject. The stopping of the CT scan may be used as the routine operation (i.e., the CT scan continues until the impedance controller 122 terminates the CT scan) or as a safety measure to prevent continuing of a CT scan which will result in low quality images.

In some embodiments of the invention, if the measured impedance values go beyond a range indicative of sufficient contrast during the CT imaging, the time of the CT imaging is extended, possibly after adding an additional dose of the contrast agent. Alternatively or additionally, the duration of the CT imaging is set responsive to the impedance when the CT imaging is initiated. For example, in accordance with this alternative, a first impedance parameter is optionally used to determine when to initiate the impedance imaging, and one or more other impedance parameters define the duration of the CT imaging. In an exemplary embodiment of the invention, the change profile of the impedance value is used in selecting the beginning time of the CT scan and the absolute impedance value is used in setting the slice width, the radiation output level and/or the CT scan duration used in the CT scan.

Referring in more detail to adjusting (210) one or more parameters of the CT imaging responsive to the impedance values, in some embodiments of the invention the energy (e.g., voltage, current (e.g., radiation output)) of the X-rays is adjusted prior to beginning the CT scan, responsive to the impedance measurements. Other parameters of the CT imaging which may be adjusted responsive to the impedance values comprise slice-width, gantry rotation speed and/or couch speed. In an exemplary embodiment of the invention, the couch speed is set responsive to the concentration of the contrast agent as detected in the impedance values, using a slower couch speed when the propagation rate of the contrast agent is slower and/or when the concentration of the contrast agent is lower. According to the couch speed, the other parameters may be adjusted. In other embodiments of the invention, the radiation output of the CT scanner and/or the slice width are set responsive to the impedance value.

For high contrast agent concentrations, a lower current and/or thinner slice width is used, thus reducing the radiation to which the patient is exposed. For lower contrast agent concentrations, a higher current and/or larger slice width is used.

The adjusted parameters may be adjusted responsive to impedance values acquired before the CT imaging begins. A single value for each parameter may be used over the entire CT scan, or the values of the parameters may change over the CT scan, based on a predetermined parameter change profile selected before the CT scan begins. Alternatively, the parameter values change responsive to impedance values determined from electrical signals acquired during the CT imaging. For example, if during the imaging the impedance values indicate a drop in the amount of contrast agent, the current or voltage of the X-rays may be increased. This alternative is optionally used with additional safety measures taken to prevent inaccurate impedance readings from causing damage. Possibly, the changes in the parameter values are taken from a limited range of values which are all safe and/or a human operator continuously monitors the imaging process and any changes in parameter values.

Other Imaging Modalities

It is noted that the CT scanner may be of any type known in the art, and may be used for substantially any imaging procedure known in the art, including cardiac CT (i.e. close-to-real-time-CT of the heart and vicinity) and angio-CT.

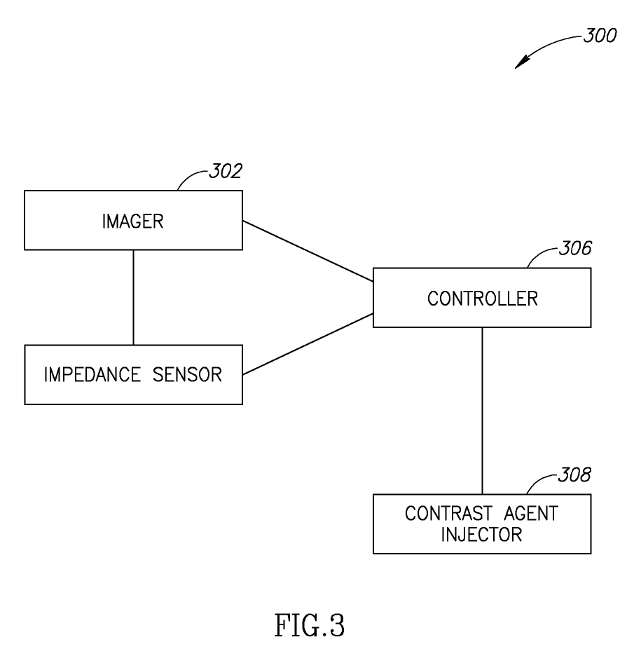

FIG. 3 is a schematic block diagram of an imaging system, in accordance with an exemplary embodiment of the invention. Imaging system 300 includes an imager 302, an impedance sensor 304, a controller 306 and a contrast agent injector 308. Imager 302 may be of substantially any modality known in the art which requires a contrast agent or other injected material, including CT imagers, magnetic resonance imagers (MRI) and nuclear medicine imagers. In some embodiments of the invention, imager 302 comprises a contrast-enhanced magnetic resonance angiography (CE-MRA) imager, for example for imaging blood flow.

Impedance sensors 304 may be of substantially any of the embodiments discussed above, including both single electrode sensors and multiple electrode sensors.

Registration

In addition to being used in setting the timing and/or parameters of the imaging modality used, in some embodiments of the invention, an impedance image may be used to provide low quality images with imaging modalities that do not provide an overall image of the patient, such as nuclear medicine. Using impedance measurements for the low quality images is relatively cheap and safe.

In parallel to generating images using a nuclear medicine modality, impedance images are optionally acquired. A fiducial marker suitable for both nuclear medicine and impedance imaging, is positioned on, or in, the patient within the image field of both the nuclear imaging and the impedance imaging. The impedance image optionally has sufficient resolution to clearly identify at least major organs of the patient, so that a physician can identify major body organs of the patient. In some embodiments of the invention, the impedance images are also used for determination of absorption coefficients of the nuclear signals in surrounding body organs.

### Exemplary Embodiment

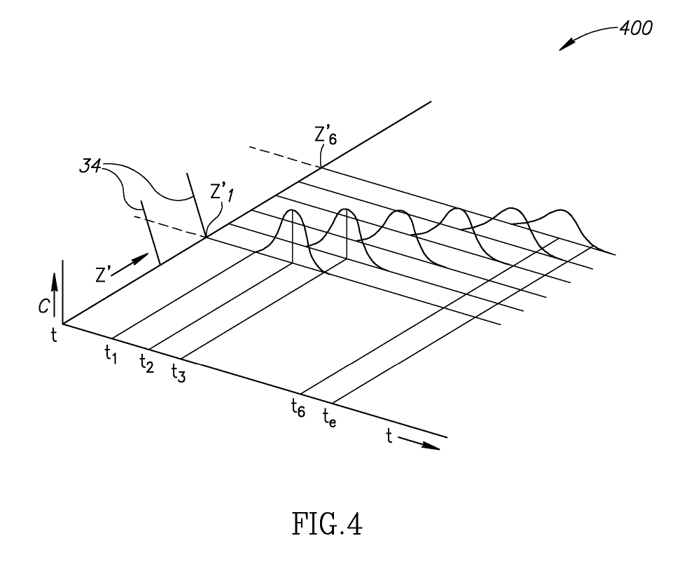

FIG. 4 is a schematic graph 400 of the concentration of a contrast agent in imaging the aorta 22 of a patient 24, as determined by EIS-scanner 120, in accordance with an exemplary embodiment of the invention. The procedure is assumed to begin at a time to, at which time the bolus of the contrast agent is administered to patient 24 and EIS-scanner 120 begins generating information responsive to the concentration of the contrast agent in patient 24.

Graph 400 has a time t-axis, a spatial z′-axis along the height of the patient and a C-axis along which values for concentration “C” of the contrast agent are indicated in arbitrary units. Coordinate z′₁ is assumed to correspond to a location at which the vena cava meets the heart and z′₆ is assumed to be located at a position at which the abdominal aorta branches to the kidneys.

At time t_{O}, CT-scan of patient 24 has not begun and all parts of the patient's body are located outside field of view 44. At a time t₁, EIS-scanner 120 determines that at a coordinate z′₁ at which the impedance signals are collected, concentration of the contrast agent begins to increase and that at a time t₂ thereafter, the concentration reaches a maximum. Optionally, EIS-controller 122 processes the time dependence of the contrast agent concentration for location z′₁ and locations in the vicinity of z′₁ to estimate a time t₃ when concentration of the contrast agent at the junction of the abdominal aorta with the descending aorta (z′₃) is expected to be optimum for CT-imaging. Controller 122 optionally also estimates a value for the concentration of the contrast agent at time t₃.

Optionally, determining time t₃ comprises estimating a value for the cardiac output of patient 24. In some embodiments of the invention, t₃ is determined responsive to a look up table (LUT) that comprises clinical data accumulated from experience in using the contrast agent for CT-imaging of patients. Optionally, t₃ is determined responsive to personal data, such as blood pressure, age, weight and/or height and/or responsive to medical history data. EIS-controller 122 transmits the time information and corresponding estimate of the optimum concentration to CT-controller 45.

Responsive to the received information, CT-controller 45 determines a current to be provided to X-ray source 32, and thereby intensity of X-rays, and begins a CT-imaging scan of patient 24. In performing the scan CT-controller 45 moves patient couch 42 to position the patient so that at time t₃ coordinate z′₃ is located in and moving through FOV 44.

According to some embodiments of the invention, as the CT-scan progresses, EIS-controller 122, constantly updates information regarding the spatial concentration of the contrast agent in the body of patient. Controller 45 uses the information to control and/or adjust CT-scan parameters of CT-scanner 20 to “chase” the bolus of contrast agent as the bolus propagates along abdominal aorta 22 and provide quality CT-imaging of abdominal aorta 22. Optionally, in providing quality CT-imaging, the CT-controller uses EIS-information to control, inter alia, motion of couch 42, rotation of rotor 36 and/or current provided to X-ray source 32.

For example, CT-controller 45 optionally uses EIS information to control translation of couch 42 so that at any given time during the CT-scan of patient 24 a location along abdominal aorta 22 at which X-ray contrast provided by the contrast agent is substantially optimum, is located in FOV 44 and hence is being CT-imaged. The translation of couch 42 is not necessarily linear, and may instead move irregularly, according to the concentration of the contrast agent.

Optionally, the CT-controller controls a hover-time of CT-scanner 20 at any given z′-coordinate responsive to the concentration of contrast agent at the coordinate as provided at least in part by electrical information. In some embodiments of the invention, the CT-controller controls current to X-ray source 32 at any given z′-coordinate responsive to electrical impedance information.

In some embodiments of the invention, CT-controller 45 determines when to end the CT-scan of patient 24 responsive to EIS-information provided by EIS-scanner 120. CT-controller 45 optionally ends the CT-scan of patient 24 at a point in time after t₆ at which EIS information indicates concentration of the contrast agent at z′₆ has decreased to a minimum level at which further exposure of the patient to X-rays does not improve quality of CT-images provided by CT-scanner 20.

It is noted that graph 400 displays EIS information as a function of a single spatial coordinate, i.e. the z′-coordinate. In other embodiments of the invention, an EIS-scanner provides CT-controller 45 with two or three-dimensional spatial information. For example, for each z′-coordinate, EIS-scanner 120 optionally provides CT-controller 45 with an extent of the concentration of contrast agent along x′ and/or y′-axes orthogonal to each other and to the z′-axis.

Region of Interest Selection

In some embodiments of the invention, impedance images are used by a physician in selecting an area of interest on which the CT imaging or other imaging modality is to focus. While impedance images may not have sufficient detail for medical analysis, the impedance images may provide sufficient detail to allow selection of the region of interest to be imaged by another modality. Selecting the region of interest based on impedance images may reduce the size of the region of interest, for example by reducing the axial extent of the CT scan, and hence the amount of radiation applied to the patient.

The region of interest to be scanned by the CT imager or other main imaging modality may be selected manually by a physician or may be selected automatically by an image analysis software. The software is optionally assigned one or more parameters, such as the size of the region to be selected.

Impedance Analysis

In some embodiments of the invention, electrical impedance scanning is used in a single imaging procedure both for acquiring medical information and for controlling parameters of another imaging modality. The medical information acquired from the impedance measurements may be acquired, for example, in accordance with any of the embodiments of U.S. Pat Nos. 6,560,480 and/or 5,810,742, mentioned above, and/or in U.S. Pat. No. 6,993,383 to Assenhiemer, the disclosure of which is incorporated herein by reference.

Alternatively to using impedance imaging for tracking the contrast agent and/or selecting the region of interest, ultrasound may be used for this purpose. Optionally, micro-bubbles are added to the contrast agent and ultrasound images are used to track the contrast agent based on the micro-bubbles therein. In other embodiments of the invention, terahertz pulse imaging (TPI), Gigahertz imaging and/or infrared imaging is used for tracking the contrast agent and/or selecting the region of interest.

It will be appreciated that the above described methods may be varied in many ways, including, changing the order of steps, and the exact implementation used.

In the description and claims of the present application, each of the verbs, “comprise” “include” and “have”, and conjugates thereof, are used to indicate that the object or objects of the verb are not necessarily a complete listing of members, components, elements or parts of the subject or subjects of the verb. It should also be appreciated that the above described methods and apparatus are to be interpreted as including apparatus for carrying out the methods and methods of using the apparatus.

The present invention has been described using non-limiting detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art. The scope of the invention is limited only by the following claims.

1. A method of imaging a living body, comprising:
administering a contrast agent to the body;
sensing signals of a first modality, which does not use ionizing radiation, from the body, responsive to the contrast agent in the body; and
acquiring one or more images of at least a portion of the body using a second imaging modality different from the first modality, wherein at least one parameter of the acquiring of the one or more images is controlled responsive to the sensed signals of the first modality.

2. A method according to claim 1, wherein the second imaging modality comprises computed tomography CT imaging.

3. A method according to claim 1, wherein the second imaging modality comprises magnetic resonance imaging MRI.

4. A method according to claim 1, wherein the second imaging modality comprises nuclear medicine imaging.

5. A method according to claim 1, wherein sensing signals of the first modality comprises sensing electrical signals.

6. A method according to claim 5, wherein sensing the electrical signals comprises sensing through an array of sensing electrodes including at least two rows and two columns of electrodes.

7. A method according to claim 5, wherein sensing the electrical signals comprises sensing using a linear array of sensing electrodes.

8. A method according to claim 5, wherein sensing the electrical signals comprises sensing using at least 10 sensing electrodes.

9. A method according to claim 1, wherein sensing signals of the first modality comprises sensing using a probe which is substantially transparent to a radiation used by the second imaging modality.

10. A method according to claim 1, comprising generating a map of an area of the body responsive to the sensed signals of the first modality and wherein the at least one parameter of the acquiring of the one or more images is controlled responsive to the generated map.

11. A method according to claim 10, comprising displaying the map.

12. A method according to claim 10, wherein the map comprises an impedance map.

13. A method according to claim 1, comprising determining a spatial concentration of the contrast agent in a plurality of points on the body and wherein the at least one parameter of the acquiring of the one or more images is controlled responsive to the determined spatial concentration.

14. A method according to claim 1, wherein the at least one parameter comprises a time parameter of the acquiring of the images using the second modality.

15. A method according to claim 14, wherein the acquiring of the one or more images is initiated responsive to the sensed signals of the first modality.

16. A method according to claim 14, wherein the acquiring of the one or more images is terminated responsive to the sensed signals of the first modality.

17. A method according to claim 10, wherein a rate of the acquiring of the one or more images is controlled responsive to the sensed signals of the first modality.

18. A method according to claim 1, wherein acquiring of the one or more images comprises radiating the patient and wherein an intensity of the radiation is selected responsive to the sensed signals of the first modality.

19. A method according to claim 1, comprising analyzing the sensed signals of the first modality to determine a medical diagnosis of the body.

20. A method of imaging a living body, comprising:
sensing electrical signals from the body;
acquiring one or more images of at least a portion of the body using an imaging modality other than impedance imaging, wherein at least one parameter of the acquiring of the one or more images is controlled responsive to the sensed electrical signals.

21. A method according to claim 20, wherein the at least one parameter comprises a boundary of a region from which the images are acquired.

22. A method according to claim 20, wherein the at least one parameter of the acquiring of the one or more images is controlled automatically by a processor responsive to the sensed electrical signals.

23. A method according to claim 20, wherein the at least one parameter of the acquiring of the one or more images is controlled by a human responsive to a display of a representation of the sensed electrical signals.

24. A method according to claim 20, wherein the at least one parameter of the acquiring of the one or more images is controlled responsive to a low quality image generated based on the sensed electrical signals.

25. A method according to claim 20, wherein the at least one parameter of the acquiring of the one or more images is controlled responsive to an attribute of a contrast agent in the body determined based on the sensed electrical signals.

26. A medical imaging apparatus, comprising:
one or more electrodes adapted for acquiring electrical signals from a subject;
an imaging unit adapted to acquire medical images of the subject, using a modality other than electrical impedance imaging; and
a controller adapted to generate an indication of a desired timing of the imaging unit, responsive to electrical signals acquired by the one or more electrodes.

27. An apparatus according to claim 26, wherein the one or more electrodes are substantially transparent to the modality of the imaging unit.

28. An apparatus according to claim 26, wherein the one or more electrodes comprises at least six electrodes.

29. An apparatus according to claim 26, comprising a syringe adapted to administer a contrast agent to the subject under control of the controller.

30. An apparatus according to claim 26, wherein the controller is adapted to determine information on a contrast agent in the subject and accordingly generate the indication of the desired timing.

31. An apparatus according to claim 26, wherein the controller is adapted to generate an indication of a desired time at which to begin an imaging session.

32. An apparatus according to claim 26, wherein the controller is adapted to generate an impedance image.

33. An apparatus according to claim 32, wherein the controller is adapted to generate an electrical impedance tomographic image.

34. A medical imaging apparatus, comprising:
one or more electrodes adapted for acquiring electrical signals from a subject;
an imaging unit adapted to acquire medical images of the subject using a modality other than electrical impedance imaging; and
a controller adapted to determine information on a contrast agent in the subject and accordingly provide suggested values for one or more parameters of the operation of the imaging unit.

35. Apparatus according to claim 34, wherein the one or more parameters comprise a beginning time of an imaging session of the imaging unit.
